(19) United States
(12) Patent Application Publication
(10) Pub. No.: US 2004/0143109 A1
(43) Pub. Date: Jul. 22, 2004

(54) Oligonucleotide probes for the detection of parodontopathogenic bacteria by in situ hybridization

(76) Inventors: Karlheinz Trebesius, Munchen (DE); Jiri Snaidr, Munchen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/638,620

(22) Filed: Aug. 11, 2003

Related U.S. Application Data
(63) Continuation of application No. PCT/EP02/01439, filed on Feb. 12, 2002.

(30) Foreign Application Priority Data
Feb. 12, 2001 (DE) 101 06 370.9

(51) Int. Cl. C07H 21/02; C07H 21/04

(52) U.S. Cl. 536/23.1

(57) ABSTRACT

The invention relates to oligonucleotide probes for the species-specific identification of parodontopathogenic bacteria by in situ hybridization. The invention further relates to oligonucleotide probe compositions used to identify such parodontopathogenic bacteria, to a method for the reliable detection of parodontopathogenic bacteria in human samples from the oral area and kits for the performance of such methods.

### RELATED APPLICATIONS

This application is a continuation and claims the benefit of priority of International Application No. PCT/EP02/064824 filed Feb. 12, 2002, designating the United States of America and published in German on Aug. 22, 2002 as WO O_{2/064824}, which claims the benefit of priority of a German Application No. 101 06 370.9 filed Feb. 12, 2001, both of which are hereby expressly incorporated by reference in their entireties.

### FIELD OF THE INVENTION

The invention relates to oligonucleotide probes for the species-specific detection of parodontopathogenic bacteria by in situ hybridization, oligonucleotide probe compositions for the detection of such parodontopathogenic bacteria, methods for the reliable detection of parodontopathogenic bacteria in human samples from the oral area and kits for the performance of such methods.

### BACKGROUND OF THE INVENTION

In spite of improvements in oral hygiene and new therapeutic procedures, parodontitis, also known as parodontosis, is still a widely spread disease. According to the 1999 German study on oral health (Deutsche Mundgesundheitsstudie), severe parodontopathies can be diagnosed in 14.1% of individuals between 35 and 44 years of age. As many as 1 in 4 individuals between 65 and 74 years of age exhibits severe parodontitis.

The progressive breakdown of the periodontium is caused by bacterial deposits, the so-called dental plaque, in the area of the tooth and its root. Without treatment, this leads ineluctably to the loss of the affected teeth. Although it used to be assumed that the increase in bacterial plaque in general is responsible for generating parodontitis (unspecific plaque hypothesis), it is meanwhile known from a series of well-substantiated studies that only a few of the well over 500 bacterial species localized in the oral cavity are associated with the development of parodontitis. Porphyromonas gingivalis, Prevotella intermedia and Bacteroides forsythus are particularly strongly involved in the initiation of this disease (Slots, J., M. Ting, 2000 Periodontol 20:82-121; Socransky, S. S. et al. 2000 Periodonto 20:341-62; Carlos, J. P. et al. 1988 J Dent Res 67:1510-4; Lai, C. H. et al. 1987 Oral Microbiol Immunol 2:152-7; Gmur, R. et al. 1989 J Periodontal Res 24:113-20). Another essential parodontopathogenic bacterium is Actinobacillus actinomycetemcomitans, which is predominantly associated with aggressive clinical courses of parodontitis (Slots, J. & M. Ting 2000 Periodontol 20:82-121).

Whereas these bacteria also occur in small numbers in healthy individuals, the disease develops when a defined threshold has been exceeded. In other words, parodontitis only develops in a host with the proper predisposition when the proportion of parodontopathogenic bacteria in the overall flora reaches a defined value.

There is now a series of possibilities available for the detection of relevant microorganisms. The detection of these bacteria in culture with artificial culture media is regarded as the standard method. This method permits both the quantification and determination of the proportion of the relevant bacteria in the culturable microflora in the parodontal sample. As however the parodontopathogenic bacteria are anaerobic and microaerophilic organisms with highly specific demands in the culture conditions, specific procedures and instruments, specifically anaerobic techniques, must be used for taking the samples, processing the material and the cultivation of these organisms. Detection in this way of the parodontal indicator bacteria by cultivation requires a lot of work and personnel and is also fairly slow, taking an average of 10 to 14 days.

The use of immunological methods is also in principal suitable for the detection of parodontopathogenic bacteria (Bonta, Y. et al. 1985 J Dent Res 64:793-8). However, the occurrence of cross-reactivities leads to frequently false positives in this method.

In principle, there are two possible ways of using the molecular biological techniques, i.e. firstly hybridization techniques, which directly detect the nucleic acids of parodontopathogenic bacteria and secondly amplification techniques (such as the polymerase chain reaction (PCR) or transcription-mediated amplification techniques (TMA)), which specifically amplify defined sections of the genetic information of parodontal indicator bacteria (Chen, C. &J. Slots 2000 Periodontol 20:53-64).

Amplification techniques permit highly sensitive and specific detection of bacteria. However, these methods are all based on enzyme-dependent amplification and exhibit a series of disadvantages, which hinder their implementation in practice:

a) Inhibitor substances present in the sample, such as the hem group of hemoglobin, can hinder or even block amplification.

b) As the hereditary material is amplified by a factor of millions, danger of cross-contaminations is high. Demanding safety measures are necessary to avoid this.

c) There are substantial expenditures on personnel and equipment.

d) Amplification techniques generally only allow qualitative and no quantitative statements.

e) Free DNA not associated with cells is also detected. In other words, the detection is positive even when the organisms to be detected are dead.

Hybridization techniques appear to be more suitable for routine studies here, as they combine robust and simple application with specific and sensitive detection. However, the major problem with hybridization techniques in connection with parodontopathogenic bacteria is that reliable quantification of the bacteria is only possible with difficulties.

An exception in this respect is the rRNA-directed in situ hybridization. If different probes are used specifically in this technique, it is not only possible to determine the number of specific microorganisms, but also their proportion in the overall flora, independently of cultivation conditions. This is fundamental for a meaningful microbial diagnosis, as a threshold value is needed to trigger parodontitis.

In addition, the detection of in situ hybridization by fluorescence provides information on the physiological state of the bacteria, on the basis of the intensity of the signal. This then serves to distinguish inactive bacteria, such as potential contaminants from other parts of the mouth, from the physiologically active subgingival flora.

A further advantage of this technique is that the bacteria can be detected in situ. The spatial association of the bacteria with each other or their colocalization with immune cells provides important insights into the pathogenesis of the parodontitis.

This method is simple and can be performed rapidly, which predestines the technique for routine use in the diagnostic laboratory or in the dental practice itself. Initial experimental results have been published on in situ hybridization for the diagnosis of parodontopathogenic microorganisms. Thus, Gersdorf et al. (1993 FEMS Immunol Med Microbiol 6: 109-14) have already been able to detect P. gingivalis and B. forsythus with fluorescently labeled probes. Moter et al. (1998 J Clin Microbiol 6:1399-403) have used this technique to detect spirochetes which are difficult or impossible to cultivate. However, the known probes for the specific detection of P. gingivalis and B. forsythus by in situ hybridization are of relatively low sensitivity.

In addition, the probe systems, which have been disclosed according to the state of the art for in situ hybridization are incomplete. Thus, no specific detection of A. actinomycetemcomitans and P. intermedia, which are important parodontopathogenic microorganisms, is possible. The specific probes which are already known for A. actinomycetemcomitans and P. intermedia, which could be used on the basis of their primary structures, are partially not suitable for the in situ hybridization technique, as binding of the probes to native ribosomal RNA is hindered by ribosomal proteins, which block the binding sites, or by blocking secondary structures in the rRNA.

In addition, the known systems based on only one hybridization probe exhibit relatively low sensitivity. Parodontopathogenic indicator bacteria containing a low number of ribosomes can therefore not, or only with difficulty, be detected with oligonucleotide probes described in the state of the art. In addition, if there is strain-strain sequence variability, the use of systems based on only one hybridization probe can lead to mispairing in the highly variable probes target regions. This gives rise to false negative results.

A further disadvantage of in situ hybridization for the detection of parodontopathogenic bacteria according to the state of the art is that, due to the low sensitivity, evaluation can only be carried out with an expensive fluorescence microscope.

### SUMMARY OF THE INVENTION

The object of the present invention is therefore to provide oligonucleotide probes which overcome the disadvantages of the state of the art and which are suitable for the in situ detection with high specificity and high sensitivity for the bacteria, which are relevant to the formation of parodontitis. A further object of the present invention is to provide a rapid and less-expensive technique for the reliable detection of the parodontal indicator bacteria in human samples from the oral cavity.

Further objectives can be derived from the following description of the invention.

### DETAILED DESCRIPTION OF THE PREFERRED EMBODIMENT

In accordance with the invention, oligonucleotide probes are provided which are suitable for the species-specific detection of parodontopathogenic bacteria of the species Actinobacillus actinomycetemcomitans, Porphyromonas gingivalis, Bacteroides forsythus and Prevotella intermedia. The sequences of the oligonucleotides according to the invention are provided in the attached sequence listing with sequences SEQ ID No. 1-17. This corresponds to the following:

SEQ ID No. 1, 2=AACT1, AACT2

SEQ ID No. 3-SEQ ID No. 5=PGIN1-PGIN3

SEQ ID No. 6-SEQ ID No. 11=BFOR1-BFOR6

SEQ ID No. 12-SEQ ID No. 17=PINT1-PINT6.

In particular, oligonucleotide probes according to the invention are provided for the species-specific detection of parodontopathogenic bacteria of the species Actinobacillus actinomycetemcomitans by in situ hybridization, wherein the oligonucleotide probes are complementary to the rRNA of Actinobacillus actinomycetemcomitans and are selected from the group consisting of:

a) A DNA sequence, comprising

| | |
|---|---|
| 5′-CAT-CAG-CGT-CAG-TAC-ATC-C-3′ | (SEQ ID NO: 1) |
| 5′-AGT-ACT-CCA-GAC-CCC-CAG-3′ | (SEQ ID NO: 2) |

or parts thereof;

b) A DNA sequence comprising the nucleic acid sequence, which hybridizes with a complementary strand of the nucleic acid sequence of a), or parts of this nucleic acid sequence;

c) A DNA sequence comprising a nucleic acid sequence, which is degenerate to a nucleic acid sequence of b), or parts of this nucleic acid sequence.

In addition, oligonucleotide probes according to the invention are provided for the species-specific detection of parodontopathogenic bacteria of the species Porphyromonas gingivalis by in situ hybridization, wherein the oligonucleotide probes are complementary to the rRNA of Porphyromonas gingivalis and are selected from the group consisting of:

a) A DNA sequence comprising

| | |
|---|---|
| 5′-CCT-CTG-TAA-GGC-AAG-TTG-C-3′ | (SEQ ID NO: 3) |
| 5′-GCG-CTC-AGG-TTT-CAC-CGC-3′ | (SEQ ID NO: 4) |
| 5′-CGG-TTA-CGC-CCT-TCA-GGT-3′ | (SEQ ID NO: 5) |

or parts thereof;

b) A DNA sequence comprising a nucleic acid sequence, which hybridizes with a complementary strand of the nucleic acid sequence of a), or parts of this nucleic acid sequence; c) A DNA sequence comprising a nucleic acid sequence, which is degenerate to a nucleic acid sequence of b), or parts of this nucleic acid sequence.

In addition, oligonucleotide probes according to the invention are provided for the species-specific detection of parodontopathogenic bacteria of the species Bacteroides forsythus by in situ hybridization, wherein the oligonucleotide probes are complementary to the rRNA of Bacteroides forsythus and are selected from the group consisting of:

a) A DNA sequence comprising

| | |
|---|---|
| 5′-GCT-ACC-ATC-GCT-GCC-CCT-3′ | (SEQ ID NO: 6) |
| 5′-CCA-TGC-GGA-ACC-CCT-GTT-3′ | (SEQ ID NO: 7) |
| 5′-CCG-CGG-ACT-TAA-CAG-CCC-ACC-T- | (SEQ ID NO: 8) |
| 3′ | |
| 5′-CGA-CAA-ACT-TTC-ACC-GCG-G-3′ | (SEQ ID NO: 9) |
| 5′-TGA-CAG-TCA-GGG-TTG-CGC-3′ | (SEQ ID NO: 10) |
| 5′-TCA-CAG-CTT-ACG-CCG-GC-3′ | (SEQ ID NO: 11) |

or parts thereof;

b) A DNA sequence comprising a nucleic acid sequence, which hybridizes with a complementary strand of the nucleic acid sequence of a), or parts of this nucleic acid sequence; c) A DNA sequence comprising a nucleic acid sequence, which is degenerate to a nucleic acid sequence of b), or parts of this nucleic acid sequence.

Finally, oligonucleotide probes according to the invention are provided for the species-specific detection of parodontopathogenic bacteria of the species Prevotella intermedia by in situ hybridization, wherein the oligonucleotide probes are complementary to the rRNA of Prevotella intermedia and are selected from the group consisting of:

a) A DNA sequence comprising,

| | |
|---|---|
| 5′-TTG-GTC-CAC-GTC-AGA-TGC-3′ | (SEQ ID NO: 12) |
| 5′-TGC-GTG-CAC-TCA-AGT-CCG-3′ | (SEQ ID NO: 13) |
| 5′-TGT-ATC-CTG-CGT-CTG-CAA-TT-3′ | (SEQ ID NO: 14) |
| 5′-CCC-GCT-TTA-CTC-CCC-AAC-3′ | (SEQ ID NO: 15) |
| 5′-CAT-CCC-CAT-CCT-CCA-CCG-3′ | (SEQ ID NO: 16) |
| 5′-TCC-CCA-TCC-TCC-ACC-GAT-GA-3′ | (SEQ ID NO: 17) |

or parts thereof;

b) A DNA sequence comprising a nucleic acid sequence, which hybridizes with a complementary strand of the nucleic acid sequence of a), or parts of this nucleic acid sequence; c) A DNA sequence comprising a nucleic acid sequence, which is degenerate to a nucleic acid sequence of b), or parts of this nucleic acid sequence.

The method of fluorescence in situ hybridization (FISH; Amann, R. I. et al. 1995 Microbial Rev 59: S. 143-169) offers a unique approach to combine the specificity of the molecular biological methods such as PCR with the possibility of visualizing the bacteria, as when antibody methods are used. This allows the highly specific identification and visualization of bacterial species, genera and groups.

The FISH technique is based on the fact that there are certain molecules in bacterial cells which possess functions which are important to life and which therefore have undergone little mutation in the course of evolution: the 16S and the 23S ribosomal ribonucleic acids (rRNA). Both are components of ribosomes, the sites of protein biosynthesis, and can serve as specific markers due to their ubiquitous distribution, their size and their structural and functional stability (Woese, C. R. 1987 Microbiol Rev 51:S. 221-271). Using comparative sequence analysis, phylogenetic relationships can be set up on the basis of these data alone. For this purpose, the sequence data must be brought into an alignment. This alignment is based on knowledge of the secondary and tertiary structure of these macromolecules and aligns the homologous positions of the ribosomal nucleic acids with each other.

Phylogenetic calculations can be performed on the basis of these data. The use of recent computer technology makes it possible to perform even large scale calculations rapidly and effectively and to set up large databases, which include the alignment sequences of the 16S-rRNA and 23S-rRNA. Rapid access to these data allows phylogenetic analysis within a short time of sequences, which have just been received. These rRNA databases can be used to construct species- and genus-specific gene probes. All available rRNA sequences are compared to each other for this purpose and probes are developed for sequences, which are specific for one bacterial species, genus or group.

In the FISH (fluorescence in situ hybridization) technique, these gene probes, which are complementary to a defined region on the ribosomal target sequence, are transformed into the cell. The gene probes are as a rule small, 16-28 bases in length, single-stranded pieces of desoxyribonucleic acid, and are directed towards a target region which is typical for the bacterial species or group. If the fluorescently-labeled gene probe finds its target sequence in a bacterial cell, it binds to it and the cells can be detected in the fluorescence microscope by their fluorescence.

The FISH analysis is generally performed on a microscope slide, which, during the evaluation of the bacteria, is visualized or made visible by irradiation with high energetic light. Alternatively, the analysis can be also performed on a microtiter plate.

In general, the methods for the specific detection of parodontopathogenic bacteria described in the present application are performed comprising the following steps:

Fixation of the bacteria contained in the sample

Incubation of the fixed bacteria with nucleic acid probe molecules according to the invention, in order to achieve hybridization,

Removal or rinsing off of non-hybridized nucleic acid probe molecules and

Detection of the bacteria hybridized with the nucleic acid probe molecules.

The nucleic acid probe can here be complementary to a chromosomal or episomal DNA, or to an mRNA or rRNA of the microorganism to be detected. It is of advantage to select a nucleic acid probe being complementary to a region, which is present in the microorganism to be detected as more than a single copy. The sequence to be detected is preferably present as 500-100,000 copies per cell, particularly preferably as 1,000 to 50,000 copies. For this reason, rRNA is used as the preferred target site, since many thousands of ribosomes, the site of protein biosynthesis, are present in every active cell. In the context of the present invention, the oligonucleotide probes according to the invention are particularly preferably directed to the 16S rRNA of the parodontopathogenic bacteria to be detected.

The nucleic acid probe in the sense of the invention can be a DNA or RNA probe, which will normally comprise 12 to 1000 nucleotides, preferably between 12 and 500, more preferably between 12 and 200 and between 12 and 100, particularly preferably between 12 and 50 and between 14 and 40 and between 15 and 30, but most preferably between 17 and 25 nucleotides. The selection of the nucleic acid probes is done according to the criteria of whether a complementary sequence is present in the microorganism to be detected. The regions selected as target sites for complementary nucleic acid probes are those which occur in the target group, for example, all strains of one species, but not in other microorganisms. For a probe consisting of 15 nucleotides 100% of the sequence should be complementary. One or several mismatches are permitted for oligonucleotides with more than 15 nucleotides.

The subject of the invention also includes modifications of the above oligonucleotide sequences, which exhibit specific hybridization with target nucleic acid sequences of the relevant bacterium, in spite of variations in sequence and/or length, and which are therefore suitable for use in a method according to the invention. These include especially

a) Nucleic acid molecules (i) being identical to any of the above oligonucleotide sequences (SEQ ID No. 1 to SEQ ID No. 17) in at least 60%, 65%, preferably in at least 70%, 75%, more preferably in at least 80%, 84%, 87% and particularly preferably in at least 90%, 94%, 96% of the bases (wherein the sequence region of the nucleic acid molecule corresponding to the sequence region of any of the oligonucleotides given above (SEQ ID No. 1 to SEQ ID No. 17) is to be considered and not the entire sequence of a nucleic acid molecule, which possibly may be longer in sequence compared to the oligonucleotides given above (SEQ ID No. 1 to SEQ ID No. 17) by one or multiple bases or (ii) differing from the above oligonucleotide sequences (SEQ ID No. 1 to SEQ ID No. 17) by one or several deletions and/or additions and which allow for specific hybridization with nucleic acid sequences of bacteria of the species Actinobacillus actinomycetemcomitans, Porphyromonas gingivalis, Bacteroides forsythus and Prevotella intermedia. “Specific hybridization” hereby means that, under the hybridization conditions described here or those known to the person skilled in the art in the context of in situ hybridization techniques, only the ribosomal RNA of the target organisms binds to the oligonucleotide and not the rRNA of non-target organisms.

b) Nucleic acid molecules, which hybridize under stringent conditions with a sequence being complementary to any of the nucleic acid molecules named under a) or to any of the probes identified in SEQ ID No. 1 to SEQ ID No. 17.

c) Nucleic acid molecules comprising an oligonucleotide sequence from SEQ ID No. 1 to SEQ ID No. 17 or comprising the sequence of a nucleic acid molecule according to a) or b) and which, in addition to these sequences or their modifications according to a) or b), have at least one further nucleotide, and which allow for specific hybridization with nucleic acid sequences of target organisms.

The degree of the sequence identity of a nucleic acid molecule with probes SEQ ID No. 1 to SEQ ID No. 17 can be determined by usual algorithms. In this respect, for example, the program for the determination of sequence identity, which is accessible under http://www.ncbi.nlm.nih.gov/BLAST (at this site there is for example the link “Standard nucleotide-nucleotide BLAST [blastn]”), is suitable here.

The nucleic acid probe molecules according to the invention can be used with various hybridization solutions in the context of the detection method. The binding of the nucleic acid probe either binds to a 100% complementary target site or to a target site with one or several mismatches, depending on whether stringent or moderate hybridization conditions are selected.

For this purpose, various organic solvents at concentrations of from 0 to 80% can be used. Moderate conditions in the sense of the invention are, for example, 0% formamide in a hybridization buffer as described in Example 1. Stringent conditions in the sense of the invention are, for example, 20 to 80% formamide in the hybridization buffer.

Parts or derivatives of nucleic acid sequences within the context of the present invention hereby mean oligonucleotide probes which may differ from the above mentioned DNA sequences according to the invention by deletion and/or addition and/or mutation or which only contain partial regions of these DNA sequences, wherein the probes retain the ability to hybridize to the specific rRNA of the above named bacteria.

In a further aspect of the present invention, an oligonucleotide probe composition is provided for the detection of pargdontopathogenic bacteria, which comprises:

i) at least one, preferably two or more oligonucleotide probes for the species-specific detection of parodontopathogenic bacteria of the species Actinobacillus actinomycetemcomitans, selected from the group consisting of:

a) A DNA sequence comprising

| | |
|---|---|
| 5′-CAT-CAG-CGT-CAG-TAC-ATC-C-3′ | (SEQ ID NO: 1) |
| 5′-AGT-ACT-CCA-GAC-CCC-CAG-3′ | (SEQ ID NO: 2) |

or parts thereof;

b) A DNA sequence comprising a nucleic acid sequence, which hybridizes with a complementary strand of the nucleic acid sequence of a), or parts of this nucleic acid sequence;

c) A DNA sequence comprising a nucleic acid sequence, which is degenerate to a nucleic acid sequence of b), or parts of this nucleic acid sequence,

and/or

ii) at least one, preferably two or more oligonucleotide probes for the species-specific detection of parodontopathogenic bacteria of the species Porphyromonas gingivalis, selected from the group consisting of

a) A DNA sequence comprising

| | |
|---|---|
| 5′-CCT-CTG-TAA-GGC-AAG-TTG-C-3′ | (SEQ ID NO: 3) |
| 5′-GCG-CTC-AGG-TTT-CAC-CGC-3′ | (SEQ ID NO: 4) |
| 5′-CGG-TTA-CGC-CCT-TCA-GGT-3′ | (SEQ ID NO: 5) |

or parts thereof;

b) A DNA sequence comprising a nucleic acid sequence, which hybridizes with a complementary strand of the nucleic acid sequence of a), or parts of this nucleic acid sequence;

c) A DNA sequence comprising a nucleic acid sequence, which is degenerate to a nucleic acid sequence of b), or parts of this nucleic acid sequence, and/or

iii) at least one, preferably two or more oligonucleotide probes for the species-specific detection of parodontopathogenic bacteria of the species Bacteroides forsythus, selected from the group consisting of

a) A DNA sequence comprising

| | |
|---|---|
| 5′-GCT-ACC-ATC-GCT-GCC-CCT-3′ | (SEQ ID NO: 6) |
| 5′-CCA-TGC-GGA-ACC-CCT-GTT-3′ | (SEQ ID NO: 7) |
| 5′-CCG-CGG-ACT-TAA-CAG-CCC-ACC-T- | (SEQ ID NO: 8) |
| 3′ | |
| 5′-CGA-CAA-ACT-TTC-ACC-GCG-G-3′ | (SEQ ID NO: 9) |
| 5′-TGA-CAG-TCA-GGG-TTG-CGC-3′ | (SEQ ID NO: 10) |
| 5′-TCA-CAG-CTT-ACG-CCG-GC-3′ | (SEQ ID NO: 11) |

or parts thereof;

b) A DNA sequence comprising a nucleic acid sequence, which hybridizes with a complementary strand of the nucleic acid sequence of a), or parts of this nucleic acid sequence;

c) A DNA sequence comprising a nucleic acid sequence, which is degenerate to a nucleic acid sequence of b), or parts of this nucleic acid sequence,

and/or

iv) at least one, preferably two or more oligonucleotide probes for the species-specific detection of parodontopathogenic bacteria of the species Prevotella intermedia, selected from the group consisting of

a) A DNA sequence comprising

| | |
|---|---|
| 5′-TTG-GTC-CAC-GTC-AGA-TGC-3′ | (SEQ ID NO: 12) |
| 5′-TGC-GTG-CAC-TCA-AGT-CCG-3′ | (SEQ ID NO: 13) |
| 5′-TGT-ATC-CTG-CGT-CTG-CAA-TT-3′ | (SEQ ID NO: 14) |
| 5′-CCC-GCT-TTA-CTC-CCC-AAC-3′ | (SEQ ID NO: 15) |
| 5′-CAT-CCC-CAT-CCT-CCA-CCG-3′ | (SEQ ID NO: 16) |
| 5′-TCC-CCA-TCC-TCC-ACC-GAT-GA-3′ | (SEQ ID NO: 17) |

or parts thereof;

b) A DNA sequence comprising a nucleic acid sequence, which hybridizes with a complementary strand of the nucleic acid sequence of a), or parts of this nucleic acid sequence;

c) A DNA sequence comprising a nucleic acid sequence, which is degenerate to a nucleic acid sequence of b), or parts of this nucleic acid sequence.

In a particularly preferred embodiment, the oligonucleotide probe composition for the detection of parodontopathogenic bacteria comprises:

i) all oligonucleotide probes for the species-specific detection of parodontopathogenic bacteria of the species Actinobacillus actinomycetemcomitans from the group consisting of

a) A DNA sequence comprising:

| | |
|---|---|
| 5′-CAT-CAG-CGT-CAG-TAC-ATC-C-3′ | (SEQ ID NO: 1) |
| 5′-AGT-ACT-CCA-GAC-CCC-CAG-3′ | (SEQ ID NO: 2) |

and/or

ii) all oligonucleotide probes for the species-specific detection of parodontopathogenic bacteria of the species Porphyromonas gingivalis from the group consisting of

| | |
|---|---|
| 5′-CCT-CTG-TAA-GGC-AAG-TTG-C-3′ | (SEQ ID NO: 3) |
| 5′-GCG-CTC-AGG-TTT-CAC-CGC-3′ | (SEQ ID NO: 4) |
| 5′-CGG-TTA-CGC-CCT-TCA-GGT-3′ | (SEQ ID NO: 5) |

and/or

iii) all oligonucleotide probes for the species-specific detection of parodontopathogenic bacteria of the species Bacteroides forsythus from the group consisting of:

| | |
|---|---|
| 5′-GCT-ACC-ATC-GCT-GCC-CCT-3′ | (SEQ ID NO:6) |
| 5′-CCA-TGC-GGA-ACC-CCT-GTT-3′ | (SEQ ID NO:7) |
| 5′-CCG-CGG-ACT-TAA-CAG-CCC-ACC-T-3′ | (SEQ ID NO:8) |
| 5′-CGA-CAA-ACT-TTC-ACC-GCG-G-3′ | (SEQ ID NO:9) |
| 5′-TGA-CAG-TCA-GGG-TTG-CGC-3′ | (SEQ ID NO:10) |
| 5′-TCA-CAG-CTT-ACG-CCG-GC-3′ | (SEQ ID NO:11) |

and/or

iv) all oligonucleotide probes for the species-specific detection of parodontopathogenic bacteria of the species Prevotella intermedia from the group consisting of:

| | |
|---|---|
| 5′-TTG-GTC-CAC-GTC-AGA-TGC-3′ | (SEQ ID NO:12) |
| 5′-TGC-GTG-CAC-TCA-AGT-CCG-3′ | (SEQ ID NO:13) |
| 5′-TGT-ATC-CTG-CGT-CTG-CAA-TT-3′ | (SEQ ID NO:14) |
| 5′-CCC-GCT-TTA-CTC-CCC-AAC-3′ | (SEQ ID NO:15) |
| 5′-CAT-CCC-CAT-CCT-CCA-CCG-3′ | (SEQ ID NO:16) |
| 5′-TCC-CCA-TCC-TCC-ACC-GAT-GA-3′ | (SEQ ID NO:17) |

In an alternatively preferred embodiment of the present invention, the composition of the oligonucleotide probes according to the invention for specifically detecting parodontopathogenic bacteria comprises all of the oligonucleotide probes according to the invention, SEQ ID No. 1-17, as given above.

Use of the oligonucleotide probe compositions according to the invention allows the highly sensitive detection of parodontal indicator germs, even when these contain only a few ribosomes. It is guaranteed in this way that the corresponding pathogens can even be detected in parodontal samples when they are in a state of low activity. The probe compositions according to the invention therefore allow for the first time the quantitative detection of parodontopathogenic bacteria in a subgingival sample, even when the number of ribosomes in the bacteria is below the threshold number, which is detectable by a simple fluorescently-labeled probe.

Moreover, in contrast to the situation with all other probes known in the state of the art, the probes described above may for example be used in combination with a dye, which stains bacteria, for the rapid determination of the proportion of a specific pathogenic bacterium in the overall microbial flora. This is of great diagnostic significance, as exceeding a critical value can lead to disease. In contrast to culture techniques, not only culturable bacteria are detected, but all bacteria present in a sample. Detection with the inventive oligonucleotide probe composition is also successful if strain variants would be present which normally differ from the strains type with respect to individual highly variable rRNA sections. Successful detection with the probes according to the invention is not only rapid, but also robust and highly specific.

A further subject of the present invention is a method for the detection of parodontopathogenic bacteria by in situ hybridization comprising the following steps:

a) Fixation of the bacteria contained in the sample;

b) Incubation of the fixed bacteria with at least one of the oligonucleotide probes of the present invention described above, preferably with one oligonucleotide probe composition according to the invention described above,

c) Detection and, optionally, quantification of the hybridized bacterial cells.

Particularly preferably, drying or filtration immobilizes the bacteria after fixation on a microscope slide.

Fixation is preferably carried out by denaturing reagents, for example, selected from the group consisting of ethanol, acetone and ethanol-acetic acid mixtures and/or crosslinking reagents, for example, selected from the group consisting of formaldehyde, paraformaldehyde and glutaraldehyde. As an alternative, fixation is carried out using heat.

In the context of the present invention, “fixing” the bacteria is generally meant to be a treatment, with which the bacterial cell envelope is made permeable for the uptake of nucleic acid probes. Ethanol is usually used for fixation. If the cell wall cannot be penetrated by nucleic acid probes after these treatments, the person skilled in the art sufficiently knows other techniques which lead to the same result. These include, for example, methanol, mixtures of alcohols, a low percentage solution of paraformaldehyde, or a diluted formaldehyde solution, enzymatic treatments or the like.

In addition, it is preferred when the oligonucleotide probes are covalently linked to a detectable marker. The detectable marker is preferably selected from the group consisting of:

a) Fluorescence marker;

b) Chemoluminescence marker;

c) Radioactive marker;

d) Enzymatically active group;

e) Hapten;

f) Nucleic acid detectable by hybridization.

The enzymatic marker is preferably selected from the group consisting of peroxidase, preferably horseradish peroxidase, and phosphatase, preferably alkaline phosphatase. The detection and quantification with the method according to the invention in this special embodiment can also be carried out with a simple light microscope. For this purpose, peroxidase-labeled oligonucleotide probes are used for the first time for in situ detection of parodontopathogenic bacteria.

This embodiment offers a series of advantages in comparison with conventionally used techniques. Firstly, a detection system based on an enzymatic reaction allows the detection of bacteria by light microscopy, which considerably reduces the purchase costs for an analytical equipment. In addition, using proper agents for counterstaining can further increase the reliability of this detection system. If conventional hematoxylin-eosin staining is carried out after in situ hybridization with peroxidase-labeled oligonucleotides, this allows not only the determination of the number and proportion of specific bacteria, but also the determination of the number of relevant immune cells and possible spatial associations with specific groups of bacteria. Moreover, improved possibilities for automating the detection system clearly arise therefrom, so that microscope-independent detection is possible. A detection system of this sort could for example be performed in microtiter plates with commercial chromogenic peroxidase substrate.

The technique described will not only bring clear facilitation in microbial diagnosis for special research laboratories and for practicing dentists, but should also lead to the latest findings in the research of this infectious disease.

In addition, it may be advantageous that the fixed cells are made permeable before incubation. During permeabilization in the sense of the present invention, holes are formed in the cell wall, although this is not destroyed as in lysis. The morphological integrity of the cell is retained. Macromolecules such as DNA, RNA and ribosomes remain in the cell. Permeabilization may be necessary, for example, to guarantee effective penetration of probes into the cell and subsequent binding to ribosomes, wherein the probes are labeled with enzyme molecules, which are large in comparison with fluorescent dyes. The permeabilization can be preferably performed by partial degradation through cell wall lytic enzymes, particularly selected from the group consisting of proteinase K, pronase, lysozyme and mutanolysin.

A further subject of the present invention is a kit for the performance of the method according to the invention described above, comprising at least one hybridization buffer as well at least one oligonucleotide probe of the present invention, preferably an oligonucleotide probe composition according to the invention.

The method described above according to the invention is an in situ hybridization method, which is based on the detection of ribosomal RNA. In a specific embodiment of the present invention described below the following steps are carried out:

Sampling;

Fixation of the sample;

Optionally, transport;

Optionally, concentration;

Immobilization of the sample on a support;

Permeabilization of the bacterial cells contained in the sample;

Hybridization of the sample;

Washing of the sample;

Detection of the hybridized probes.

The clinical experience of the practicing physician is decisive in the selection of the patients or affected areas of the teeth. The comments in 1998 of the Society for Parodontology and the Germany Society of Odontology, Oral and Maxillo Sciences (Deutsche Gesellschaft für Zahn-, Mund- und Kieferheilkunde) on microbiological diagnosis in marginal parodontitis can serve as guideline. Samples can either be taken from individual parodontal pockets or “pooled samples” from several subgingival sites. Alternatively, the technique described can be used to examine also supragingival sites or other samples from the oral-pharyngeal area (e.g., saliva samples). Samples from subgingival sites are either taken with specific dental instruments (e.g., curettes, scalers and the like) or special paper tips, preferably ISO45 from the Alfred Becht Company (Offenburg, Germany) or other manufacturers.

The bacteria, which have been sampled by different methods are then transferred to a suitable fixation medium, to kill the bacteria and to hinder the degradation of ribosomal RNA. In principle, either denaturing reagents, such as ethanol, acetone or ethanol-acetic acid mixtures may be used, or crosslinking reagents, such as formaldehyde, paraformaldehyde or glutaraldehyde. It is also possible to use mixtures from both groups of fixatives (e.g. ethanol together with formaldehyde).

The extracted bacteria can also be eluted directly into a drop of water present on a microscope slide. The bacteria are then fixed by heating on an open flame, such as a Bunsen burner, or in a temperature-controlled incubator, e.g., at 80° C., fixed and simultaneously immobilized on a microscope slide. Fixed samples can be stored without further special precautions or equipment and may be transported, possibly.

If no heat fixation was carried out, the fixed samples are then immobilized on a microscope slide by drying. Alternatively, filtration procedures can be used for immobilization. Using a membrane filter, even large sample volumes can be applied on a filter. Polycarbonate membranes are preferably used, which are then hybridized in an analogous manner to the immobilized samples on the microscope slides.

Optionally, the immobilization can be followed by treatment with increasing concentrations of ethanol (e.g. 50%, 80% and 96% ethanol for 3 minutes each).

If large marker molecules are used (e.g. horseradish peroxidase, alkaline phosphatase, i.a.), further permeabilization of the bacterial cell walls may be advantageous, to guarantee effective diffusion of the labeled probe molecules into the bacterial cell. Various cell wall lytic enzymes can be used, e.g. proteinase K, pronase, lysozyme, mutanolysin and the like. In the method according to the invention for the detection of parodontopathogenic bacteria, A. actinomycetemcomitans, P. gingivalis, B. forsythus and P. intermedia, the enzymes proteinase K and lysozyme in the form shown in example 3 are best suited for the permeabilization of the cell walls. However, various chemical reagents (e.g., 1N HCl or detergents) can also be used for permeabilization of individual bacterial cells. Another series of increasing concentrations of ethanol is used to stop the enzyme reaction.

In accordance with the invention, the nucleic acid probe is incubated with the microorganism which has been fixed in the above sense, to allow penetration of the nucleic acid probe molecules into the microorganism and hybridization of nucleic acid probe molecules with the nucleic acids of the microorganism. The non-hybridized nucleic acid probe molecules are then removed by the usual washing steps.

The specifically hybridized nucleic acid probe molecules can then be detected in the corresponding cells. The prerequisite for this is that the nucleic acid probe molecule is detectable, e.g., in that the nucleic acid probe molecule is covalently linked to a marker. Detectable markers which are used and which are all well known to the person skilled in the art include fluorescent groups such as, for example, CY2 (available from Amersham Life Sciences, Inc., Arlington Heights, USA), CY3 (also available from Amersham Life Sciences), CY5 (also available from Amersham Life Sciences), FITC (Molecular Probes Inc. Eugene, USA), FLUOS (available from Roche Diagnostics Ltd, Mannheim, Germany), TRITC (available from Molecular Probes Inc. Eugene, USA), 6-FAM or FLUOS-PRIME. Chemical markers, radioactive markers or enzymatic markers, such as horseradish peroxidase, acid phosphatase, alkaline phosphatase and peroxidase can be used as well. A series of chromogens is known for each of these enzymes, which can be reacted instead of the natural substrate, forming colored or fluorescent products. Examples of such chromogens are given in the following table.

TABLE 1

| Enzyme | Chromogen |
|---|---|
| 1. Alkaline | 4-methylumbelliferylphosphate (*), |
| phosphatase | bis(4-methyiumbelliferylphosphate), (*) 3-O- |
| and acid | methylfluorescein, flavone-3- |
| phosphatase | diphosphate triammonium salt (*), |
| | p-nitrophenylphosphate disodium salt |
| 2. Peroxidase | tyramine hydrochloride (*), 3-(p-hydroxyphenyl)- |
| | propionic acid(*), p-hydroxyphenethylalcohol(*), |
| | 2,2′-azino-di-3-ethylbenzthiazolinesulfonic acid |
| | (ABTS), ortho-phenylendiamine dihydrochloride, |
| | o-dianisidine, 5-aminosalicylic acid, p-ucresol |
| | (*), 3,3′-dimethyloxybenzidine, 3-methyl-2- |
| | benzothiazoline hydrazone, tetramethylbenzidine |
| 3. Horseradish | H₂O₂ + diammonium benzidine |
| peroxidase | H₂O₂ + tetramethylbenzidine |
| 4. β-D-galactosidase | o-Nitrophenyl-β-D-galactopyranoside, |
| | 4-methylumbelliferyl-β-D-galactoside |
| 5. Glucose oxidase | ABTS, glucose and thiazolyl blue |

*Fluorescence.

Finally, it is possible to form the nucleic acid probe molecules in such a way that there is a further nucleic acid sequence at the 5′- or 3′-end, which is also suitable for hybridization. This nucleic acid sequence in turn includes approx. 15 to 1000, preferably 15 to 50 nucleotides. This second nucleic acid region can then be recognized by an oligonucleotide probe, which is detectable by any of the agents given above.

Another possibility is the coupling of the detectable nucleic acid probe molecule to a hapten. After the nucleic acid probe molecule has been released from the target nucleic acid, the isolated nucleic acid probe molecule can be brought into contact with antibodies, which recognize the hapten. An example of such a hapten is digoxigenin or its derivatives. Apart from the given examples, the person skilled in the art is also very familiar with further examples.

The standard hybridization method is performed on microscope slides, on filters, on a microtiter plate or in a reaction vessel. The analysis depends on the type of labeling of the used probe and can be conducted using an optical microscope, an epifluorescence microscope, chemoluminometer, fluorometer, flow cytometer or the like.

Particularly preferably, the kit of the present invention contains the following specific probes for the detection of parodontopathogens:

Probes which detect strains of the species Actinobacillus actinomycetemcomitans:

| | |
|---|---|
| AACT1: | |
| 5′-CAT-CAG-CGT-CAG-TAC-ATC-C-3′ | (SEQ ID NO:1) |
| AACT2: | |
| 5′-AGT-ACT-CCA-GAC-CCC-CAG-3′ | (SEQ ID NO:2) |

Probes which detect strains of the species Porphyromonas gingivalis:

| | |
|---|---|
| PGIN1: | |
| 5′-CCT-CTG-TAA-GGC-AAG-TTG-C-3′ | (SEQ ID NO:3) |
| PGIN2: | |
| 5′-GCG-CTC-AGG-TTT-CAC-CGC-3′ | (SEQ ID NO:4) |
| PGIN3: | |
| 5′-CGG-TTA-CGC-CCT-TCA-GGT-3′ | (SEQ ID NO:5) |

Probes which detect strains of the species Bacteroides forsythus:

| | |
|---|---|
| BFOR1: | |
| 5′-GCT-ACC-ATC-GCT-GCC-CCT-3′ | (SEQ ID NO:6) |
| BFOR2: | |
| 5′-CCA-TGC-GGA-ACC-CCT-GTT-3′ | (SEQ ID NO:7) |
| BFOR3: | |
| 5′-CCG-CGG-ACT-TAA-CAG-CCC-ACC-T-3′ | (SEQ ID NO:8) |
| BFOR4: | |
| 5′-CGA-CAA-ACT-TTC-ACC-GCG-G-3′ | (SEQ ID NO:9) |
| BFOR5: | |
| 5′-TGA-CAG-TCA-GGG-TTG-CGC-3′ | (SEQ ID NO:10) |
| BFOR6: | |
| 5′-TCA-CAG-CTT-ACG-CCG-GC-3′ | (SEQ ID NO:11) |

Probes which detect strains of the species Prevotella intermedia:

| | |
|---|---|
| PINT1: | |
| 5′-TTG-GTC-CAC-GTC-AGA-TGC-3′ | (SEQ ID NO:12) |
| PINT2: | |
| 5′-TGC-GTG-CAC-TCA-AGT-CCG-3′ | (SEQ ID NO:13) |
| PINT3: | |
| 5′-TGT-ATC-CTG-CGT-CTG-CAA-TT-3′ | (SEQ ID NO:14) |
| PINT4: | |
| 5′-CCC-GCT-TTA-CTC-CCC-AAC-3′ | (SEQ ID NO:15) |
| PINT5: | |
| 5′-CAT-CCC-CAT-CCT-CCA-CCG-3′ | (SEQ ID NO:16) |
| PINT6: | |
| 5′-TCC-CCA-TCC-TCC-ACC-GAT-GA-3′ | (SEQ ID NO:17) |

The probe molecules according to the invention may be used within the scope of the detection method with various hybridization solutions. Different organic solvents at concentrations of from 0% to 80% can be used. For example, formamide is preferably used at a concentration of from 20% to 60%, particularly preferably at a concentration of 20% in the hybridization buffer. In addition, the hybridization buffer contains a salt, preferably sodium chloride, at a concentration of 0.1 mol/l to 1.5 mol/l, preferably of 0.5 mol/l to 1.0 mol/l, more preferably of 0.7 mol/l to 0.9 mol/l and most preferably of 0.9 mol/l. The hybridization buffer may be buffered with various compounds, such as Tris/HCl, sodium citrate, PIPES or HEPES buffer, which are used in the concentration range of 0.01 mol/l to 0.1 mol/l, preferably from 0.01 mol/l to 0.08 mol/l and particularly preferably at 0.02 mol/l. The pH usually lies between 6.0 and 9.0, preferably between 7.0 and 8.0. Preferably, the hybridization buffer contains 0.02 mol/l Tris/HCl at pH 8.0.

In addition, detergents, such as Triton X or sodium dodecyl sulphate (SDS) at a concentration of 0.001% to 0.2%, preferably from 0.005% to 0.1%, are present. Here, a particularly preferred hybridization buffer contains 0.01% SDS.

Other additives can be used for various experimental questions, such as unlabeled nucleic acid fragments (e.g. fragmented salmon sperm DNA, unlabeled oligonucleotides, and others) or molecules which can accelerate the hybridization reaction due to a reduction in the reaction volume (polyethyleneglycol, polyvinylpyrrolidone, dextran sulfate and others). These additives are added by the person skilled in the art at the known and conventional concentrations to the hybridization buffer.

It is to be understood that the person skilled in the art can select the given concentrations of the components of the hybridization buffer in such a way that the required stringency of the hybridization reaction is achieved. Particularly preferred embodiments reflect stringent to particularly stringent hybridization conditions. Using these stringent conditions, the person skilled in the art can determine whether a given nucleic acid molecule permits the species-specific detection of nucleic acid sequences of parodontopathogenic bacteria and can therefore be used reliably in the context of the invention.

It is obvious that the person skilled in the art can select the given concentrations of the components of the hybridization buffer in such a way that the required stringency of the hybridization reaction is achieved. Particularly preferred embodiments reflect stringent to particularly stringent hybridization conditions. Using these stringent conditions, the person skilled in the art can establish whether a given nucleic acid molecule allows the specific detection of nucleic acid sequences of target organisms and can therefore be used reliably in the context of the invention. If required, the person skilled in the art is in a position to reduce or increase the stringency by changing the parameters of the hybridization buffer, depending on the probe and the target organism.

The concentration of the nucleic acid probe in the hybridization buffer depends on the type of labeling and the number of target structures. To allow rapid and efficient hybridization, the number of nucleic acid probe molecules should exceed the number of target structures by several orders of magnitude. On the other hand, it needs to be considered when working with fluorescence in situ hybridization (FISH) that an excessively high level of fluorescently-labeled nucleic acid probe molecules leads to an increase in background fluorescence. The concentration of the nucleic acid probe molecules should therefore be in the range of 0.5-500 ng/μl, preferably between 1.0-100 ng/μl and particularly preferably between 1.0-50 ng/μl.

In the context of the method of the present invention, the preferred concentration is 1-10 ng of each nucleic acid probe molecule used per μl hybridization solution. The used volume of hybridization solution should be between 8 μl and 100 ml; in a particularly preferred embodiment of the method of the present invention it is 30 μl.

The duration of the hybridization is normally between 10 minutes and 12 hours; the hybridization is preferably carried out for about 1.5 hours. The hybridization temperature is preferably between 44° C. and 48° C., particularly preferably 46° C., whereby the parameter of the hybridization temperature as well as the concentration of salts and detergents in the hybridization solution can be optimized based on the nucleic acid probes, in particular their lengths and the degree of complementarity to the target sequence in the cell to be detected. The person skilled in the art is familiar with the applicable calculations.

After completion of the hybridization, the non-hybridized and excess nucleic acid probe molecules should be removed or rinsed off, which is usually performed by a conventional washing solution. If desired, this washing solution can contain 0.001-0.1% of a detergent such as SDS, preferably 0.005-0.05%, particularly preferably 0.01%, and Tris/HCl at a concentration of 0.001-0.1 mol/l, preferably 0.01-0.05 mol/l, particularly preferably 0.02 mol/l, wherein the pH of the Tris/HCl is in the range of 6.0 to 9.0, preferably at 7.0-8.0, particularly preferably at 8.0. A detergent can be included, but is not absolutely necessary. The washing solution also usually contains NaCl, at a concentration, depending on the necessary stringency, of from 0.003 mol/l to 0.9 mol/l, preferably from 0.01 mol/l to 0.9 mol/l. The NaCl concentration is particularly preferably about 0.215 mol/l.

In addition, the washing solution may contain EDTA, at a preferred concentration of 0-0.005 mol/l. The washing solution can further contain current preservatives at quantities familiar to the person skilled in the art.

In general, buffer solutions are used in the washing step, which can in principle be very similar to the hybridization buffer (buffered sodium chloride solution), but with the provision that the washing step is usually performed in a buffer at lower salt concentrations or at higher temperature. The following equation can be used for the theoretical estimation of the hybridization conditions:

Td=81.5+16.6 1g[Na⁺]+0.4×(% GC)−820/n−0.5 X(% FA)

Td=dissociation temperature in ° C.

[Na⁺]=molarity of sodium ions

% GC=proportion of guanine and cytosine nucleotides relative to the number of total bases

n=hybrid length

% FA=formamide content

Using this equation, for example, the proportion of formamide in the washing buffer (which should be kept as low as possible because of formamide's toxicity) can be replaced with a correspondingly lower content of sodium chloride. However, the person skilled in the art is aware, on the basis of the extensive literature on in situ hybridization methods, that and how these components can be varied. All that was said above with respect to the hybridization conditions also applies to the stringency of the hybridization conditions.

The “washing” of the unbound nucleic acid probe molecules is normally performed at temperatures in the range of 44° C. to 52° C., preferably at 44° C. to 50° C., and particularly preferably at 46° C. for a duration of 10-40 minutes, preferably for 15 minutes.

In an alternative embodiment of the method of the present invention, the nucleic acid molecules according to the invention are used in the so-called Fast-FISH method for specifically detecting the given target organisms. The Fast-FISH method is known to the person skilled in the art and is, for example, described in German patent applications DE 199 36 875 and WO 99/18234. It is hereby specifically referred to the disclosure in these documents for performing the detection methods described in them.

Furthermore, kits according to the invention for the performance of the corresponding methods are made available. The hybridization arrangement contained in these kits is, for example, described in the German patent application 100 61 655.0. It is hereby specifically referred to these documents, with respect to their disclosure of the in situ hybridization arrangement described in them.

Apart from the described hybridization arrangement (called VIT reactor), the most important component of the kits is the respective hybridization solution with the specific nucleic acid probe molecules for the microorganisms to be detected, as described above (so-called VIT solution). The kits also always contain the corresponding hybridization buffer (corresponding to the hybridization solution without the probe molecules) and a concentrate of the corresponding washing solution. The kit may also contain fixation solutions (50% ethanol, absolute ethanol), if needed, and an embedding solution (finisher), if needed. Finishers are commercially available and their activity also includes the prevention of rapid bleaching of fluorescent probes under the fluorescent microscope. Optionally, solutions for parallel performing a positive control and a negative control may also be contained.

The following examples are intended to describe the invention, however, without limiting it:

### EXAMPLE 1

### Specific Detection of A. actinomycetemcomitans, P. gingivalis, B. forsythus and P. intermedia

To prove the specificity of the samples of the present invention, a number of reference organisms was ordered from publicly accessible bacterial culture collections and cultivated on the media recommended by the culture collections. As soon as colonies were visible on the culture media, a colony was picked from the plate with an inoculating loop and suspended in a fixation solution (4% formaldehyde in 1×PBS). The optimal optical density of the bacterial suspension is 0.2, measured at a wavelength of 600 nm. The bacteria were left in the fixation solution for between 1 and 24 hours and were then sedimented for 5 min at 8,000 rpm (Rotina 35, Rotor type 1714, Hettich, Tuttlingen). The supernatant was discarded and the pellet was washed in 1×PBS (initial volume). After another centrifugation step (same conditions as above), the cells were taken up in a 1:1 mixture of EtOH/PBS and stored at −20° C. until use.

5 μl were taken from this suspension and applied to the wells of a Teflon-coated microscope slide, air-dried and treated with serially increasing ethanol concentrations (50%, 80% and 100% for 3 minutes each). The immobilized samples were then air-dried and 10 μl of the hybridization buffer (0.9 mol/l NaCl, 0.02 mol/l Tris/HCl, pH 8.0, 0.01% SDS, 20% formamide, 5 ng of each hybridization probe AACT1 and AACT2, PGIN1-3, BFOR1-6, PINT1-6) were added. To test whether the corresponding reference cells contain enough rRNA in order to be detected by this method, not only a Cy3-labelled specific probe, but also a FLUOS-labeled universal probe were included in the hybridization.

The hybridization was performed in a humidity chamber, which was equilibrated with hybridization buffer. The time of hybridization was at least 90 minutes. After this, the unbound probe was removed by placing the hybridized microscope slide in a 50 ml tube containing the washing buffer (0.215 mol/l NaCl, 0.02 mol/l Tris/HCl, pH 8.0, 0.01% SDS) and was incubated for 15 minutes at 48° C.

The hybridized microscope slides were coated with a suitable embedding medium and then analyzed by fluorescence microscopy.

Table 2 shows the reference strains used and the results obtained with the probes of the present invention.

a) Specificity of the AACT probes:

| | | | | Eub |
|---|---|---|---|---|
| | | | | 338- |
| Organism | Strain | AACT1 | AACT2 | FLU |
| Haemophilus | DSM 11123 | + | + | + |
| actinomycetemcomitans | | | | |
| Haemophilus | DSM 8324 | + | + | + |
| actinomycetemcomitans T | | | | |
| Haemophilus influenzae | DSM 4690 | − | − | + |
| Haemophilus parainfluenzae | DSM 8978 | − | − | + |
| Haemophilus ducreyi | DSM 8925 | − | − | + |
| Haemophilus parasuis | ATCC 19417 | − | − | + |
| Haemophilus aphrophilus | ATCC 33389 | − | − | + |
| Haemophilus paraphrophilus | ATCC 29241 | − | − | + |
| Pasteurella avium | ATCC 29546 | − | − | + |
| Mannheimia haemolytica | DSM 10531 | − | − | + |
| Porphyromonas gingivalisT | ATCC 33277 | − | − | + |
| Porphyromonas | DSM 20707 | − | − | + |
| asaccharolytica | | | | |
| Prevotella melaninogenica | | − | − | + |
| Prevotella intermedia | DSM 20706 | − | − | + |
| Prevotella bivia | GH 1029 | − | − | + |
| Bacteroides uniformis | GH 1077 | − | − | + |
| Bacteroides vulgatus | DSM 1447 | − | − | + |
| Bacteroides ureolyticus | | − | − | + |
| Veilonella parvula | | − | − | + |
| Bacteroides ovatus | DSM 1896 | − | − | + |
| Bacteroides fragilis | ATCC 25295 | − | − | + |

b) Specificity of the PGIN probes:

| Organism | Strain | PGIN1 | PGIN2 | PGIN3 | EUB338 |
|---|---|---|---|---|---|
| Porphyromonas gingivalis | DSM 20709 | + | + | + | + |
| Porphyromonas gingivalis | ATCC 33277 | + | + | + | + |
| Porphyromonas | DSM 20707 | − | − | − | + |
| assaccharolyticus | | | | | |
| Porphyromonas endodontalis | ATCC 35406 | − | − | − | + |
| Porphyromonas catoniae | ATCC 51270 | − | − | − | + |
| Bacteroides forsythus | ATCC 43037 | − | − | − | + |
| Prevotella bivia | GH 1029 | − | − | − | + |
| Prevotella intermedia | DSM 20704 | − | − | − | + |
| Bacteroides fragilis | ATCC 25295 | − | − | − | + |
| Bacteroides uniformis | | − | − | − | + |
| Bacteroides vulgatus | ATCC 29327 | − | − | − | + |
| Haemophilus | DSM 11123 | − | − | − | + |
| actinomycetemcomitans | | | | | |
| Haemophilus influenzae | DSM 4690 | − | − | − | + |
| Haemophilus parainfluenzae | DSM 8978 | − | − | − | + |
| Clostridium paraputrefaciens | GH 2151 | − | − | − | + |
| Clostridium cadaveris | GH 2141 | − | − | − | + |
| Mannheimia haemolytica | DSM 10531 | − | − | − | + |

c) Specificity of the BFOR probes:

| | | BFOR1, 2, 3, | |
|---|---|---|---|
| Organism | Strain | 4, 5 and 6 | EUB338 |
| Bacteroides forsythus | ATCC 43037 | + | + |
| Porphyromonas gingivalis | ATCC 33277 | − | + |
| Porphyromonas | DSM 20707 | − | + |
| assaccharolyticus | | | |
| Porphyromonas | ATCC 35406 | − | + |
| endodontalis | | | |
| Porphyromonas | ATCC 51270 | − | + |
| catoniae | | | |
| Capnocytophaga | 21334 | − | + |
| ochraceae | | | |
| Prevotella intermedia | DSM 20706 | − | + |
| Prevotella loeschei | GH 1068 | − | |
| Prevotella melaninogenica | GH 1061 | − | |
| Prevotella bivia | GH 1029 | − | |
| Prevotella ruminicola | GH 914 | − | + |
| ssp. ruminicola | | | |
| Prevotella ruminicola | GH 1024 | − | + |
| ssp. brevis | | | |
| Prevotella corporis | GH 830 | − | + |
| Prevotella disiens | GH 1015 | − | + |
| Prevotella heparinolytica | GH 918 | − | + |
| Bacteroides distasonis | GH 872 | − | + |
| Bacteroides uniformis | GH 1077 | − | + |
| Bacteroides ovatus | GH 1048 | − | + |
| Bacteroides vulgatus | ATCC 29327 | − | + |
| Actinobacillus | DSM 11123 | − | + |
| actinomycetemcomitans | | | |

d) Specificity of the PINT probes:

| | | PINT1, 2, 3, | |
|---|---|---|---|
| Organism | Strain | 4, 5 and 6 | EUB338 |
| Prevotella intermedia | DSM 20707 | + | + |
| Prevotella intermedia | GH 1084 | + | + |
| Prevotella intermedia | GH 1030 | + | + |
| Prevotella intermedia | GH 1032 | + | + |
| Prevotella intermedia | GH 1052 | + | + |
| Prevotella bivia | GH 1029 | − | + |
| Prevotella loeschei | GH 1068 | − | + |
| Prevotella melaninogenica | GH 1061 | − | + |
| Prevotella ruminicola | GH 914 | − | + |
| ssp. ruminicola | | | |
| Prevotella ruminicola | GH 1024 | − | + |
| ssp. brevis | | | |
| Prevotella corporis | GH 830 | − | + |
| Prevotella disiens | GH 1015 | − | + |
| Prevotella dentalis | DSM 3688 | − | + |
| Prevotella bryantii | DSM 11371 | − | + |
| Prevotella nigrescens | DSM 13386 | − | + |
| Prevotella buccae | DSM 20615 | − | + |
| Prevotella heparinolytica | GH 918 | − | + |
| Porphyromonas gingivalis | ATCC 33277 | − | + |
| Porphyromonas | DSM 20707 | − | + |
| assaccharolyticus | | | |
| Porphyromonas endodontalis | CCUG 29541 | − | + |
| Porphyromonas catoniae | CCUG 41358 | − | + |
| Bacteroides ovatus | GH 1048 | − | + |
| Bacteroides forsythus | CCUG 33226 | − | + |
| Bacteroides vulgatus | ATCC 29327 | − | + |
| Bacteroides uniformis | GH 1077 | − | + |
| Bacteroides distasonis | B98-026006/3 | − | + |
| | (GH 872) | | |
| Bacteroides fragilis* | ATCC 25295 | − | + |
| Haemophilus | DSM 11123 | − | + |
| actinomycetemcomitans | | | |

DSM: Deutsche Sammlung von Mikroorganismen (German Collection of Microorganisms)
ATCC: American Type Culture Collection
CCUG: Culture Collection, University of Gothenburg
GF: Max von Pettenkofer Institute, Culture Collection, branch Grosshadern

### EXAMPLE 2

### Detection of Parodontopathogenic Bacteria in Samples From Patients With Parodontitis

The parodontal samples were taken either with a scaler or with a sterile paper tip specifically intended for this purpose. If a scaler was used, after the bacterial plaque has been removed from the gums pocket, the scaler was stirred in the fixation solution (4% formaldehyde solution in 1×PBS) for long enough until the bacterial deposits (plaque) sticking to it are fully suspended in 200 μl fixation solution. If the sterile paper tips were used for sampling, these were to be removed aseptically from the packaging, and, after the patient was pre-treated (drying of the corresponding site, removal of supragingival plaque), were introduced into the parodontal pocket from which the sample was to be taken. The paper tip was left there for 10-20 seconds, removed and transferred into a test-tube containing 200 μl fixation solution. The paper tip was sent to the test laboratory under this condition. 1/10 of the volume of a 1% solution of Triton X-100 was then mixed with the fixation solution and shaken well for 2×30 seconds, in order to elute the bacteria from the paper tip.

After this, centrifugation was carried out for 5 minutes at 8,000 rpm, the pellet was washed as described in Example 1 and the bacteria were finally transferred into 60 μl of a 1:1 mixture of ethanol and PBS. The bacteria in the sample can be stored in this solution at −20° C. for at least 3 months.

5 μl of this suspension were applied to a microscope slide and hybridized with the probes according to the invention, as given in detail in Example 1. After hybridization, the samples were stained with DAPI (4′, 6-diamidino-2-phenylindoldihydrochloride; Sigma; Deisenhofen; Germany), which binds unspecifically to DNA. The samples were then overlaid with a PBS solution containing 1 μg/ml DAPI, and incubated for 5-15 minutes in the dark at room temperature. After a further washing step with 1×PBS, the samples could be analyzed in a suitable embedding medium (Citifluor AF1, Citifluor Ltd., London, UK; Vectashild, Vector Laboratories, Burlingame, U.S.A), using a fluorescence microscope.

The quantitative evaluation was performed via a counting ocular, according to the instructions of the microscope manufacturer (Zeiss, Oberkochen, Germany). Table 3 shows the quantitative evaluation of three different patient samples.

TABLE 3

| Running | Pocket | Aac | | Pgi | | Bfo | | Pint | |
|---|---|---|---|---|---|---|---|---|---|
| No. | Depth | Proportion | No. | Prop. | No. | Prop. | No. | Prop. | No. |
| 1 | 10 mm | | n.d. | 49.7% | 2.7 × 10⁷ | | n.d. | | n.d. |
| 2 | 8 mm | | n.d. | 21.7% | 1.75 × 10⁶ | 19.7% | 1.6 × 10⁶ | 4.8% | 4.0 × 10⁵ |
| 3 | 8 mm | | 10²-10³ | 6.2% | 1.1 × 10⁶ | 8.8% | 1.5 × 10⁶ | | 2 × 10⁵ |

n.d.: no data

### EXAMPLE 3

### Detection of Parodontopathogenic Bacteria With Oligonucleotide Probes, Which Are Labeled With Horseradish Peroxidase

The parodontal samples were taken, fixed and immobilized on the microscope slides, as described in Example 2. After this, the cells present in the sample were permeabilized by an incubation time of 15 minutes in a 10 μg/ml proteinase K solution or in a 250 μg/ml lysozyme solution. The enzymatic reaction was stopped by adding an ethanol series at increased concentrations (50%, 80%, 100%, for 3 minutes each).

The hybridization was performed as described in Example 1, but with a buffer containing 40% formamide instead of 20% formamide. In addition, the hybridization was performed at 35° C. After 90 minutes, the microscope slide was removed from the humid chamber and incubated for 15 minutes at 37° C. in a washing buffer-POD (0.056 mol/l NaCl; 0.05 mol/l EDTA, 0.02 mol/l Tris/HCl, pH 8.0; 0.01% SDS). The hybridized sample was then overlaid for 10 minutes with a substrate solution containing diaminobenzidine. This solution was prepared by dissolving a tablet containing diaminobenzidine and a tablet containing H₂O₂ from the SIGMA FAST DAB Tablet Sets (D4168) in 1 ml substrate buffer (0.15 mol/l NaCl; 0.1 mol/l Tris/HCl, pH 8.0). After the tablets had fully dissolved in the substrate buffer, 10 μl of this ready substrate solution was applied to the hybridized samples and incubated for 10 minutes at room temperature. The sample was then rinsed with 1×PBS and was examined under the microscope, either immediately or after suitable counterstaining.

HE staining was suitable for counterstaining and this was prepared in the following manner. The moist, hybridized microscope slides were immersed into a glass cuvette filled with hemalaun (Merck, Germany, product no. 1.09249.0500). After 3-5 minutes, the microscope slides were rinsed for a short time in distilled water and then exposed to cold running tap water for 10 minutes for the blue color to develop. The microscope slide was then immersed for 3-5 minutes into a cuvette containing eosin. The microscope slides were then rinsed for a short time in 90% ethanol and then in absolute ethanol. The microscope slide was finally immersed in three different xylene baths, until the xylene solution remained clear.

What is claimed is:

1. An oligonucleotide probe for a species-specific detection of parodontopathogenic bacteria by in situ hybridization, said probe selected from the group consisting of:
i) SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17;
ii) oligonucleotides being identical to any of the oligonucleotides from i) to at least 80% of the bases and allowing for specific hybridization with nucleic acid sequences of parodontopathogenic bacteria of the species Actinobacillus actinomycetemcomitans, Porphyromonas gingivalis, Bacteroides forsythus and/or Prevotella intermedia;
iii) oligonucleotides differing from any of the oligonucleotides from i) and ii), in that they are at least one nucleotide longer, and
iv) oligonucleotides hybridising with a sequence, which is complementary to any oligonucleotide from i), ii) and iii), under stringent conditions.

2. An oligonucleotide probe composition for the detection of parodontopathogenic bacteria by in situ hybridization, comprising:
i) at least one oligonucleotide probe for the species-specific detection of parodontopathogenic bacteria of the species Actinobacillus actinomycetemcomitans, said oligonucleotide probe selected from the group consisting of:
a) a DNA sequence, comprising: SEQ ID NO: 1; SEQ ID NO: 2; or parts thereof; and
b) a DNA sequence, comprising a nucleic acid sequence, which hybridizes with a complementary strand of the nucleic acid sequence of a) under stringent conditions, or parts of this nucleic acid sequence,
and/or
ii) at least one oligonucleotide probe for the species-specific detection of parodontopathogenic bacteria of the species Porphyromonas gingivalis, selected from the group consisting of:
a) a DNA sequence, comprising: SEQ ID NO: 3; SEQ ID NO: 4; SEQ ID NO: 5; or parts thereof, and
b) a DNA sequence comprising a nucleic acid sequence, which hybridizes with a complementary strand of the nucleic acid sequence of a), or parts of this nucleic acid sequence,
and/or
iii) at least one oligonucleotide probe for the species-specific detection of parodontopathogenic bacteria of the species Bacteroides forsythus, selected from the group consisting of:
a) a DNA sequence, comprising SEQ ID NO: 6, SEQ ID NO: 7, SEQ ID NO: 8, SEQ ID NO: 9, SEQ ID NO: 10, SEQ ID NO: 11, or parts thereof, and
b) a DNA sequence, comprising a nucleic acid sequence, which hybridizes with a complementary strand of the nucleic acid sequence of a), or parts of this nucleic acid sequence,
and/or
iv) at least one oligonucleotide probe for the species-specific detection of parodontopathogenic bacteria of the species Prevotella intermedia, selected from the group consisting of:
a) a DNA sequence, comprising SEQ ID NO: 12, SEQ ID NO: 13, SEQ ID NO: 14, SEQ ID NO: 15, SEQ ID NO: 16, SEQ ID NO: 17, or parts thereof, and
b) a DNA sequence, comprising a nucleic acid sequence, which hybridizes with a complementary strand of the nucleic acid sequence of a), or parts of this nucleic acid sequence.

3. The oligonucleotide probe composition according to claim 2, comprising:
i) all oligonucleotide probes for the species-specific detection of parodontopathogenic bacteria of the species Actinobacillus actinomycetemcomitans, selected from the group consisting of: a DNA sequence, comprising SEQ ID NO: 1, and SEQ ID NO: 2; and/or
ii) all oligonucleotide probes for the species-specific detection of parodontopathogenic bacteria of the species Porphyromonas gingivalis, selected from rom the group consisting of: SEQ ID NO: 3, SEQ ID NO: 4, and SEQ ID NO: 5; and/or
iii) all oligonucleotide probes for the species-specific detection of parodontopathogenic bacteria of the species Bacteroides forsythus, selected from the group consisting of: SEQ ID NO: 6, SEQ ID NO: 7, SEQ ID NO: 8, SEQ ID NO: 9, SEQ ID NO: 10, and SEQ ID NO: 1; and/or
iv) all oligonucleotide probes for the species-specific detection of parodontopathogenic bacteria of the species Prevotella intermedia, selected from the group consisting of: SEQ ID NO: 12, SEQ ID NO: 13, SEQ ID NO: 14, SEQ ID NO: 15, SEQ ID NO: 16, and SEQ ID NO: 17.

4. The oligonucleotide probe composition according to claim 2, comprising all oligonucleotide probes of SEQ ID No. 1-17.

5. A method for adetection of parodontopathogenic bacteria in a sample by in situ hybridization, comprising:
fixing the parodontopathogenic bacteria contained in the sample,
incubating the fixed bacteria with at least one oligonucleotide probe according to claim 1, in order to achieve hybridization, and
detecting the parodontopathogenic bacterial cells with the hybridized oligonucleotide probes.

6. The method of claim 5, wherein said parodontopathogenic bacterial cells are also quantified.

7. The method according to claim 5, wherein the bacteria are immobilized on a support after fixation.

8. The method according to claim 7, wherein the bacteria are immobilized by drying or filtration.

9. The method according to claim 5, wherein said fixing is performed by a denaturing reagent.

10. The metjof of claim 9, wherein said denaturing reagent is selected from the group consisting of: ethanol, acetone and ethanol-acetic acid mixtures.

11. The method according to claim 5, wherein said fixing is performed by a cross-linking reagent.

12. The methof of claim 11, wherein said ross-linking reagent is selected from the group consisting of formaldehyde, paraformaldehyde and glutaraldehyde.

13. The method according to claim 5, wherein said fixing is performed by heat fixation.

14. The method according to claim 5, wherein the oligonucleotide probes are covalently linked to a detectable marker.

15. The method according to claim 14, wherein said detectable marker is selected from the group consisting of: a fluorescence marker, a chemoluminescence marker, a radioactive marker, an enzymatic marker, a hapten, and a nucleic acid detectable by hybridization.

16. The method according to claim 15, wherein the enzymatic marker is selected from the group consisting of peroxidase and phosphatase.

17. The method of claim 16, wherein said peroxidase is horseradish peroxidase and said phosphatase is alkaline phosphatase.

18. The method according to claim 5, wherein the fixed cells are made permeable before incubation.

19. The method according to claim 18, wherein the fixed cells are made permeable by partial degradation by cell wall lytic enzymes.

20. The method according to claim 19, wherein the cell wall lytic enzymes are selected from the group consisting of: proteinase K, pronase, lysozyme and mutanolysin.

21. The method according to claim 5, wherein the parodontopathogenic bacteria are bacteria of the species Actinobacillus actinomycetemcomitans, Porphyromonas gingivalis, Bacteroidesforsythus and/or Prevotella intermedia.

22. The method according to claim 5, wherein the oligonucleotide is selected from the group consisting of: SEQ ID NO: 1 and SEQ ID NO: 2.

23. The method according to claim 5, wherein the oligonucleotide is selected from the group consisting of: SEQ ID NO: 3, SEQ ID NO: 4, and SEQ ID NO: 5.

24. The method according to claim 5, wherein the oligonucleotide is selected from the group consisting of: SEQ ID NO: 6, SEQ ID NO: 7, SEQ ID NO: 8, SEQ ID NO: 9, SEQ ID NO: 10, and SEQ ID NO: 11.

25. The method according to claim 5, wherein the oligonucleotide is selected from the group consisting of: SEQ ID NO: 12, SEQ ID NO: 13, SEQ ID NO: 14, SEQ ID NO: 15, SEQ ID NO: 16, and SEQ ID NO: 17.

26. A kit for carrying out the method for the detection of parodontopathogenic bacteria in a sample by in situ hybridization, said kit comprising at least one oligonucleotide probe according to claim 1.

27. A kit according to claim 26, further comprising a hybridization solution.
